(19) United States
(12) Patent Application Publication
(10) Pub. No.: US 2023/0240263 A1
(43) Pub. Date: Aug. 3, 2023

(54) PAW CLEANING APPARATUS

(71) Applicant: Maria Stella Romualdo, Plantation, FL (US)

(72) Inventor: Maria Stella Romualdo, Plantation, FL (US)

(21) Appl. No.: 17/587,341

(22) Filed: Jan. 28, 2022

(51) Int. Cl. A01K 13/00; A46B 13/00; A46B 13/04; A46B 15/00; A61L 2/10

(52) U.S. Cl. CPC A01K 13/001; A01K 13/002; A46B 13/001; A46B 13/04; A46B 15/0006; A46B 15/0008; A61L 2/10; A46B 2200/1093; A61L 2202/17; A61L 2202/14

(57) ABSTRACT

An apparatus for cleaning a pet's paw includes a cylindrical container for holding water and/or cleaning fluid, a cap configured to cover the top opening and to accept insertion of the pet's paw, a cylindrical brush element, a base for holding the cylindrical container, an electrical motor configured to interface with the cylindrical brush element so as to spin the cylindrical brush element to clean the pet's paw, a controller configured to control the electrical motor, the controller configured to activate the electrical motor to spin in the clockwise and counterclockwise directions, to move upwards and downwards along the central longitudinal axis, and to move radially away from and towards the central longitudinal axis, and one or more UV lights located within the cylindrical container, the one or more UV lights configured to sanitize by emitting UV light.

### CROSS-REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The claimed subject matter relates to the field of home appliances and, more specifically, the claimed subject matter relates to the field of home appliances for pets.

### BACKGROUND

A common problem associated with owning dogs or cats is that these pets tend to carry dirt, soil, filth, excrement and/or mud into one's home after taking a walk outdoors. Dirt and debris easily enter into the gaps within a pet's paws, and it can be challenging to remove it. To generally prevent dirt, soil, mud, filth, excrement, and/or bacteria from being introduced into one's home, animal paws must be cleaned before permitting the animal to enter the home. A typical method of cleaning an animal's paws may include manually washing each paw by hand with wet and/or dry towel. But because dirt and debris can get impacted within the gaps of a pet's paw, it can be difficult to remove said dirt and debris. Therefore, it can be time-consuming and tedious to manually wash a pet's paws.

Additionally, bathing a small pet such as a dog or a cat is a common chore often performed by a pet's owners. Pet washing is undertaken indoors using a conventional bathtub or basin using soapy water, or outdoors using a tub, basin and/or garden hose. Various conventional devices and apparatus have been used for the complete washing of a pet. One such conventional device is a hose nozzle that sprays water at a high pressure. These conventional devices, however, are not be particularly adapted for a thorough cleaning of the paws of a pet, which has many cracks and crevices within which dirt and debris can be lodged. Additionally, these conventional devices are not readily transportable and storable between uses. Furthermore, these conventional pet washing devices are not readily adaptable for daily use, do not provide rapid and effective cleaning of pet paws, and cannot be readily cleaned, sealed and stored between frequent uses.

Therefore, what is needed is a system and method for improving the problems with the prior art, and more particularly for a more expedient, simple and efficient method and system for cleaning pet's paws before being allowed to enter a home.

### BRIEF SUMMARY

In one embodiment, an apparatus for cleaning a pet's paw is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claim subject matter's scope.

The apparatus for cleaning a pet's paw includes a cylindrical container for holding water and/or cleaning fluid, the cylindrical container having a top opening; a cap configured to cover the top opening of the cylindrical container, the cap further including an orifice configured to accept insertion of the pet's paw while preventing water and/or cleaning fluid from exiting the cap; a cylindrical brush element located within the cylindrical container, the brush element including a plurality of bristles that extend towards a central longitudinal axis, wherein an interior volume of the cylindrical brush element is configured to accept the pet's paw; a base for holding the cylindrical container, the base including a handle; an electrical motor configured to interface with the cylindrical brush element so as to spin the cylindrical brush element to clean the pet's paw, the electrical motor further configured to spin the cylindrical brush element in a clockwise and counterclockwise direction, to move the cylindrical brush element upwards and downwards along the central longitudinal axis, and to move the cylindrical brush element radially away from and towards the central longitudinal axis; a controller configured to control the electrical motor, the controller configured to activate the electrical motor to spin in the clockwise and counterclockwise directions, to move upwards and downwards along the central longitudinal axis, and to move radially away from and towards the central longitudinal axis; and one or more ultraviolet (UV) lights located within the cylindrical container, the one or more UV lights configured to sanitize by emitting UV light.

Additional aspects of the claimed subject matter will be set forth in part in the description which follows, and in part will be obvious from the description, or may be learned by practice of the claimed subject matter. The aspects of the claimed subject matter will be realized and attained by means of the elements and combinations particularly pointed out in the appended claims. It is to be understood that both the foregoing general description and the following detailed description are exemplary and explanatory only and are not restrictive of the disclosed subject matter, as claimed.

### BRIEF DESCRIPTION OF THE SEVERAL VIEWS OF THE DRAWINGS

The accompanying drawings, which are incorporated in and constitute part of this specification, illustrate embodiments of the claimed subject matter and together with the description, serve to explain the principles of the claimed subject matter. The embodiments illustrated herein are presently preferred, it being understood, however, that the claimed subject matter is not limited to the precise arrangements and instrumentalities shown, wherein:

FIG. 1 is an illustration of a top perspective view of the pet paw cleaning apparatus, in accordance with one embodiment.

FIG. 2 is an illustration of a top perspective view of a close up of the pet paw cleaning apparatus, in accordance with one embodiment.

FIG. 3 is an illustration of a top perspective view of the pet paw cleaning apparatus with a cutout, in accordance with one embodiment.

FIG. 4A is an illustration of another top perspective view of the pet paw cleaning apparatus with a cutout, in accordance with one embodiment.

FIG. 4B is an illustration showing directions pertinent to the pet paw cleaning apparatus, in accordance with one embodiment.

FIG. 5 is an illustration of another top perspective view of the pet paw cleaning apparatus with a cutout, in accordance with one embodiment.

FIG. 6 is an illustration of a top perspective view of a close up of the pet paw cleaning apparatus, in accordance with one embodiment.

FIG. 7 is an illustration of a pet using the pet paw cleaning apparatus, in accordance with one embodiment.

### DETAILED DESCRIPTION

It should be understood that the embodiments disclosed herein are only examples of the many advantageous uses of the innovative teachings of the claimed embodiments. In general, statements made in the specification of the present application do not necessarily limit any of the various claimed embodiments. Moreover, some statements may apply to some inventive features but not to others. In general, unless otherwise indicated, singular elements may be in the plural and vice versa with no loss of generality. In the drawing like numerals refer to like parts through several views.

The disclosed embodiments improve over the prior art by providing a pet paw cleaning apparatus that addresses the issues with the prior art by providing a pet paw cleaning device that is quick and easy to use, as well as portable and efficient in its purpose. The claimed embodiments also provide a higher level of cleaning and completes said cleaning in a record time. Additionally, the claimed device is also easy to handle and to utilize, as well as easy to store and clean in between uses. Said features increase usability and proper usage of the pet paw cleaning devices on a daily basis. The disclosed embodiments also improve over the prior art by providing a pet paw cleaning apparatus that reduces or eliminates the ability of dirt and debris from entering into a person's home due to their pet's unclean paws. The disclosed embodiments further improve over the prior art by permitting the claimed apparatus to be held at an inclination whereby naturally allowing the animal's paw to be positioned in a relaxed extension rather than an upright standing position thereby resulting in a more comfortable and relaxed position of both the dog and the person holding the apparatus as there is no tendency of the animal to either put its weight on the paw being cleansed or tire the person with the weight of the dog supported only by the handle. The disclosed embodiments provide greater proficiency in cleaning between the pads of the paw, as well as the sides and the top of the paw, which were not properly addressed by the prior art. The circulating bristles of the claimed embodiments reach the top, sides, front, and back of the paw in their entirety.

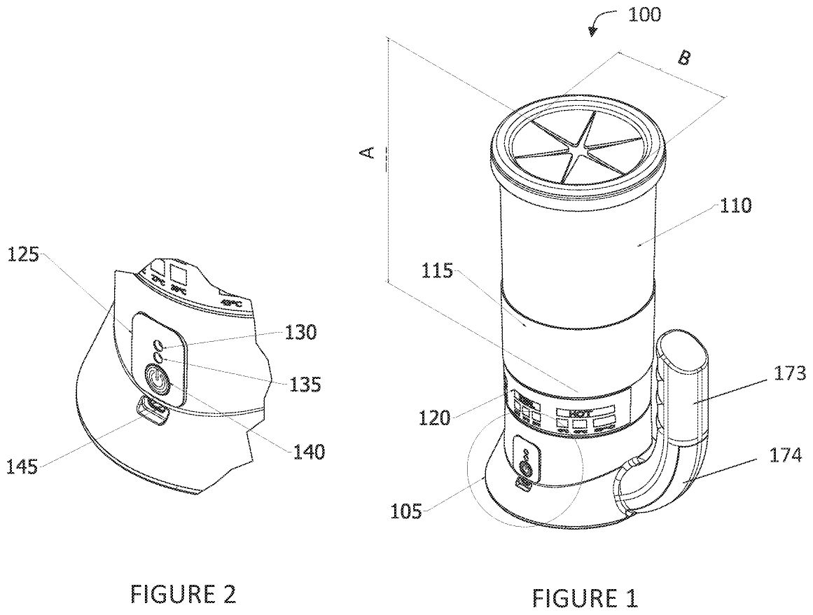

Referring now to the drawing figures in which like reference designators refer to like elements, there is shown in FIG. 1 is an illustration of a top perspective view of the pet paw cleaning apparatus 100, in accordance with one embodiment. FIG. 2 is an illustration of a close up of the user interface section 125 of the pet paw cleaning apparatus 100, in accordance with one embodiment. FIG. 1 shows that the apparatus 100 for cleaning a pet's paw includes a cylindrical container 110 (of height A) for holding water and/or cleaning fluid, the cylindrical container having a top opening and a cap 150 configured to cover the top opening of the cylindrical container. In one embodiment, the cylindrical container 110 is composed of a clear material, such as clear plastic, acrylic or glass, and said container may include one or more measurement markings on its exterior or interior surface, such as markings for measuring volume (such as milliliters, ounces) or for measuring how much water to put into the container (such as a fill line). In one embodiment, the cylindrical container 110 includes a bottom portion 115 that is covered with another material, such as a rubber material, wherein said material aids in the user holding the apparatus.

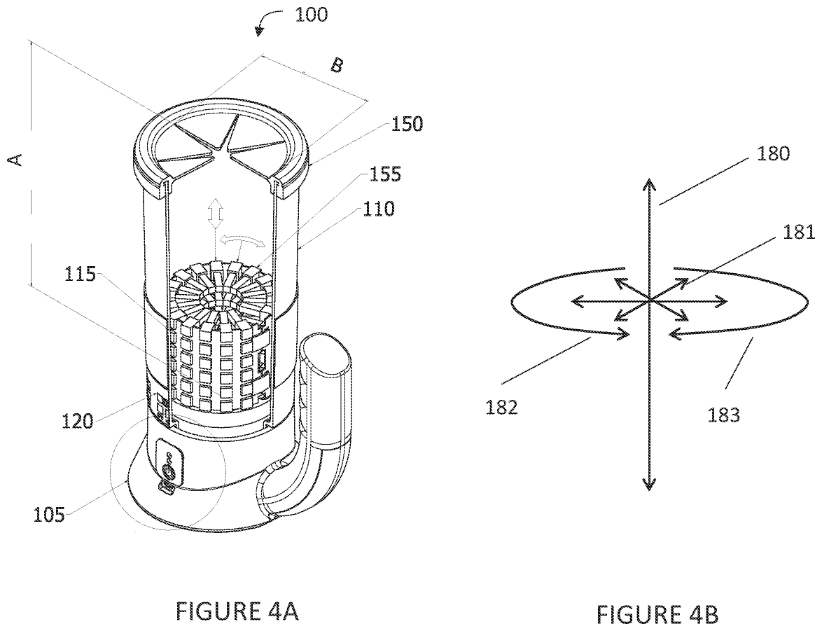

The cap 150 further includes an orifice (of width B) configured to accept insertion of the pet's paw 175 while preventing water and/or cleaning fluid from exiting the cap (see FIGS. 4A, 5 and 7). The cap 150 comprises an orifice, and a plurality of flaps 151 that extend towards a central point of the cap. The cap may allow an insertion of an animal's paws through a silicone lid opening having star shaped flaps. For instance, there may be more or less six flaps crisscrossing a proximate center portion of the silicone cap, forming a star shaped opening. While the device is powered on, the cap may prevent water from splashing out while the brush spins in circular motion to clean the animal's paws. The apparatus may be tilted almost horizontally without water spilling out the cap. In extreme cases, with a really difficult pet, if the apparatus is tilted completely horizontally, some water may slowly spill out the cap, but may still maintain enough water inside the cup to still function properly to clean the animal's paws.

FIG. 1 further shows the apparatus 100 includes a base 105 for holding the cylindrical container 110, the base including a handle 174. The base 105 may have a bottom portion that is wider than a narrower top portion, such that the width of the base gradually becomes narrower from the bottom potion to the top portion. The handle 174 may extend upwards from the bottom portion of the base 105, such that the handle 174 extends upwards in a direction parallel to the central longitudinal axis 180 of the apparatus 100. The handle 174 may further include a top portion 173 that is an ergonomic element that includes shapes and contours that fit the human hand, which allows a user to easily hold the apparatus 100 during use. The handle may be used to easily maneuver the apparatus around an animal's paw with one hand and hold the animal with the other hand. The handle may have a very comfortable grip portion 173 which may enable a comfortable hold with five or less fingers and palm. The handle may allow a user to easily and effectively maneuver the device even if a difficult animal is getting washed.

FIG. 2 further shows the user interface section 125 of the pet paw cleaning apparatus 100. The user interface section 125 may include one or more LED lights 130, 135 that may indicate a variety of functions, such as whether the apparatus is on or off, whether the apparatus is currently spinning, the current status of the battery, the charging status of the battery, etc. The user interface section 125 may include an on-off button 140 that is used to turn the apparatus on or off, begin the apparatus spinning, change the spinning mode of the apparatus, etc. The user interface section 125 may include a charging port 145 (such as a USB port) that is used to recharge the rechargeable battery of the apparatus 100.

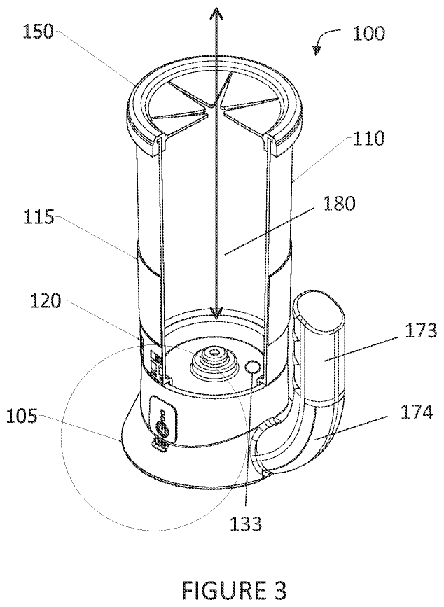

FIG. 3 is an illustration of a top perspective view of the pet paw cleaning apparatus 100 with a cutout, in accordance with one embodiment. FIG. 3 includes a cutout in the cylindrical container 110, thereby showing the interior of the container. FIG. 3 shows the central longitudinal axis 180 of the apparatus 100 as it extends via the central axis of the cylindrical container 110. FIG. 3 also shows one or more ultraviolet (UV) lights 133 located within the cylindrical container, the one or more UV lights configured to sanitize by emitting UV light. UV light is a form of electromagnetic radiation with wavelength from 10 nm (with a corresponding frequency around 30 PHz) to 400 nm (750 THz). UV light may be used as a disinfection method to kill or inactivate microorganisms by destroying nucleic acids and disrupting their DNA, leaving them unable to perform vital cellular functions. UV light can be used to make a surface an inhospitable environment to microorganisms such as bacteria, viruses, molds, and other pathogens. The light emitted by ultraviolet (UV) lights 133 passes through the cylindrical brush element 155 with positioning and strength sufficient to sanitize the paw while also not causing harm to the animal's or the human's eyes.

FIG. 4A is an illustration of another top perspective view of the pet paw cleaning apparatus 100 with a cutout, in accordance with one embodiment. FIG. 4A shows a cylindrical brush element 155 located within the cylindrical container, the brush element 155 including a plurality of bristles that extend towards the central longitudinal axis 181, wherein an interior volume of the cylindrical brush element 155 is configured to accept the pet's paw 175. Note that the plurality of bristles that extend horizontally or laterally towards the central longitudinal axis 181. The brush element may have frayed ends to aid in the cleaning process. In other embodiments, the brush may include a rubber material, plastic material, silicone material, etc.

In one embodiment, the bristles of the brush element 155 may extend further up the container. The longer bristled brush element 155 permits the paw to be cleaned by the spinning bristled brush element 155 completely, as opposed to the prior art wherein the dog had to remain flat-footed on top of the brush which can be very annoying for most dogs due to the sensitivity of the areas between the pads of their paws. With the dog's paw positioned in the prone position with the toes pointing down in a natural and relaxed position, the gentle brushes of the brush element 155 softly sweep dirt and debris away without causing anxiety or any pressure on the pads or toenails.

FIG. 4B is an illustration showing directions pertinent to the pet paw cleaning apparatus 100, in accordance with one embodiment. Said directions describe the movement of the motor and, by extension, the brush element during use. FIG. 4B shows the central longitudinal axis 180 of the apparatus 100 as it extends via the central axis of the cylindrical container 110. FIG. 4B also shows a series of directions 181 that show a series of arrows pointing radially away from the central longitudinal axis 180 of the apparatus 100. The series of directions 181 are all perpendicular to the central longitudinal axis 180. FIG. 4B also shows a clockwise direction 183 and a counterclockwise direction 182, which extend around the central longitudinal axis 180.

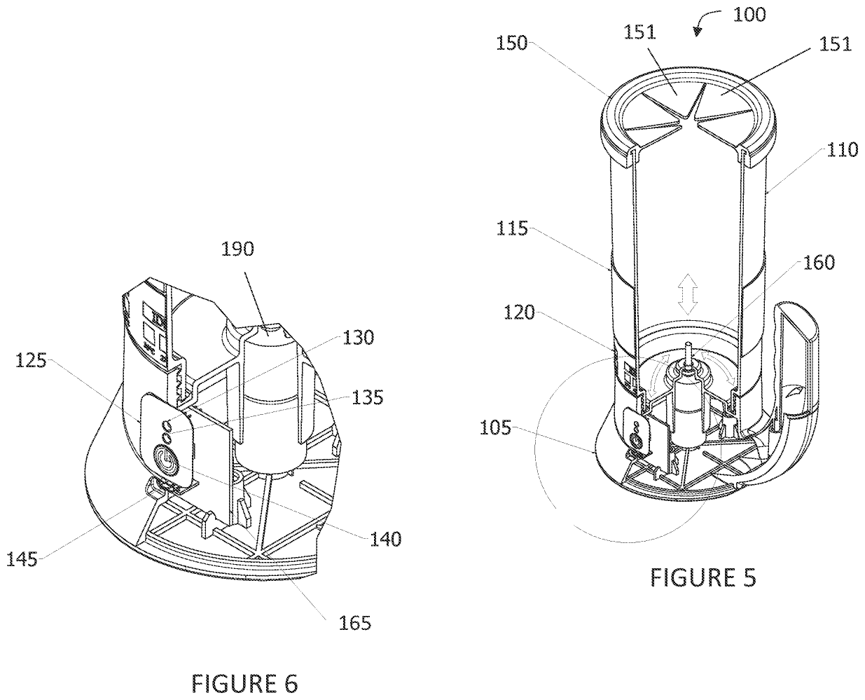

FIG. 5 is an illustration of another top perspective view of the pet paw cleaning apparatus 100 with a cutout, in accordance with one embodiment. FIG. 5 shows that the apparatus 100 includes a spindle 160 that is connected to an electrical motor 190 configured to interface with the cylindrical brush element 155 so as to spin the cylindrical brush element to clean the pet's paw 175. The electrical motor 190 is further configured to spin the cylindrical brush element in a clockwise 183 and counterclockwise 182 direction, to move the cylindrical brush element upwards and downwards along the central longitudinal axis 180, and to move the cylindrical brush element radially 181 away from and towards the central longitudinal axis. Said movement of the electrical motor 190 in said directions also moves the spindle and, by extension, the brush element in the same directions.

FIG. 5 shows that the apparatus 100 includes a controller 165 configured to control the electrical motor 190, the controller configured to activate the electrical motor to spin in the clockwise and counterclockwise directions, to move upwards and downwards along the central longitudinal axis, and to move radially away from and towards the central longitudinal axis. The controller may be a processor, a programmable logic controller, an ASIC, a PCB, a PCBA or the like. In one embodiment, the controller 165 is configured to switch between activating the electrical motor to spin in the clockwise and counterclockwise directions, activating the electrical motor to move upwards and downwards along the central longitudinal axis, and activating the electrical motor to move radially away from and towards the central longitudinal axis, an external button on the base, the button configured to activate and deactivate the controller

In one embodiment, the electrical motor 190 is conductively coupled to a rechargeable battery that is charged via the charging port 145. In one embodiment, the electrical motor 190 is conductively coupled to a timer configured to activate and deactivate the controller at predefined periods of time. In one embodiment, the electrical motor 190, the controller 165, the timer, the buttons and lights 140, 130, 135 are all communicatively coupled. The timer may enable a user to not have to turn off the apparatus, instead use both hands, and not let go of a difficult animal who might try to get away. As such, the timer may facilitate in the cleaning process.

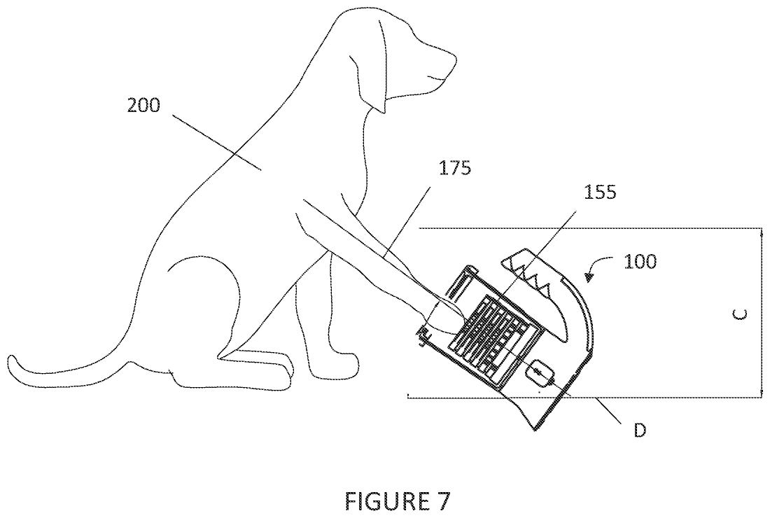

FIG. 7 is an illustration of a pet or dog 200 using the pet paw cleaning apparatus 100, in accordance with one embodiment. FIG. 7 shows that the apparatus 100 includes the cylindrical brush element 155 located within the cylindrical container, the brush element 155 including a plurality of bristles that extend towards the central longitudinal axis 181, wherein an interior volume of the cylindrical brush element 155 is configured to accept the pet's paw 175. FIG. 7 shows that the apparatus 100 may be used in a small area D, and that the apparatus 100 may be tilted such that is exhibits an effective tilted height C during use.

The pet paw cleaning apparatus 100 can be held at an inclination whereby naturally allowing the animal's paw 175 to be positioned in a relaxed extension rather than an upright standing position thereby resulting in a more comfortable and relaxed position of both the dog and the person holding the apparatus 100 as there is no tendency of the animal to either put its weight on the paw being cleansed or tire the person with the weight of the dog supported only by the handle. The pet paw cleaning apparatus 100 is proficient in cleaning between the pads of the paw, as well as the sides and the top of the paw, since the circulating bristles of the apparatus 100 reach the top, sides, front, and back of the paw in their entirety.

In one embodiment, one or more elements of the apparatus 100 may be composed of a plastic, such as polyolefin, polyacrylate, polystyrene, polyamide, polyvinyl alcohol, poly(alkylene acrylate), poly(ethylene vinyl alcohol), poly(alkylene vinyl acetate), polyurethane, polyacrylonitrile, polyester, fluoropolymer, polycarbonate, or combinations thereof. In one embodiment, one or more elements of the apparatus 100, or a portion thereof, may comprise a surface that is ink-printable, i.e., the surface allows for ink printing on its surface. In another embodiment, one or more elements of the apparatus 100, or a portion thereof, may be opaque, transparent, semi-transparent, or translucent. In another embodiment, one or more elements of the apparatus 100 may be composed of at least one of a thermoplastic, a thermosetting polymer, polyethylene, polypropylene, polystyrene, polyvinyl chloride, polytetrafluoroethylene (PTFE), polystyrene, polyvinyl chloride, nylon, polyester, polyethylene terephthalate, high density polyethylene, polyvinylidene chloride, high impact polystyrene, or mixtures thereof. One or more elements of the apparatus 100 may further be composed of any moldable plastic, ABS plastic, injection grade plastic, bio-plastic or biodegradable plastic. In another embodiment, one or more elements of the apparatus 100, or any portion thereof, may be composed of rubber or a similar type of polymer.

In one embodiment, brush 155 may be composed of a polymeric foam such as EVA foam, LDPE foam, nitrile rubber foam, neoprene, polyimide foam, polypropylene foam, polystyrene foam, styrofoam, polyurethane foam, PVC foam, silicone foam or microcellular foam. The material of brush 155 must be soft enough so as not to irritate or harm the animal, yet sufficiently flexible to allow for the spinning bristles to be inserted between the animal's pads as well as the fur, nails, and pads without causing irritation, harm, or alarm.

In another embodiment, one or more elements of the apparatus 100, or any portion thereof, may be composed of stainless steel, iron, silver, platinum, gold, zinc, copper, nickel, or any alloys or combinations of the above. The composition of the one or more elements of the apparatus 100, or any portion thereof, may be mixed with harder metals for strength and durability.

Embodiments of the invention, for example, are described above with reference to block diagrams and/or operational illustrations of methods, systems, and processes. While certain embodiments have been described, other embodiments may exist. Further, the disclosed processes may be modified in any manner, including by reordering stages and/or inserting or deleting stages, without departing from the invention.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. An apparatus for cleaning a pet's paw, the apparatus comprising:
a) a cylindrical container for holding water and/or cleaning fluid, the cylindrical container having a top opening;
b) a cap configured to cover the top opening of the cylindrical container, the cap further including an orifice configured to accept insertion of the pet's paw while preventing water and/or cleaning fluid from exiting the cap;
c) a cylindrical brush element located within the cylindrical container, the brush element including a plurality of bristles that extend towards a central longitudinal axis, wherein an interior volume of the cylindrical brush element is configured to accept the pet's paw;
d) a base for holding the cylindrical container, the base including a handle;
e) an electrical motor configured to interface with the cylindrical brush element so as to spin the cylindrical brush element to clean the pet's paw, the electrical motor further configured to spin the cylindrical brush element in a clockwise and counterclockwise direction, to move the cylindrical brush element upwards and downwards along the central longitudinal axis, and to move the cylindrical brush element radially away from and towards the central longitudinal axis;
f) a controller configured to control the electrical motor, the controller configured to activate the electrical motor to spin in the clockwise and counterclockwise directions, to move upwards and downwards along the central longitudinal axis, and to move radially away from and towards the central longitudinal axis; and
g) one or more ultraviolet (UV) lights located within the cylindrical container, the one or more UV lights configured to sanitize by emitting UV light.

2. The apparatus of claim 1, wherein the cylindrical container is a clear container.

3. The apparatus of claim 2, wherein the cap comprises a plurality of flaps that extend towards a central point of the cap.

4. The apparatus of claim 3, wherein the plurality of bristles are composed of a foam material.

5. The apparatus of claim 4, wherein the base comprises a bottom side that is wider than a top side.

6. The apparatus of claim 5, wherein the electrical motor is conductively coupled to a rechargeable battery.

7. The apparatus of claim 6, wherein the controller is configured to switch between activating the electrical motor to spin in the clockwise and counterclockwise directions, activating the electrical motor to move upwards and downwards along the central longitudinal axis, and activating the electrical motor to move radially away from and towards the central longitudinal axis.

8. The apparatus of claim 7, wherein the handle comprises a shaft that extends upwards parallel to the central longitudinal axis.

9. The apparatus of claim 8, further comprising an external button on the base, the button configured to activate and deactivate the controller.

10. The apparatus of claim 9, further comprising a timer communicatively coupled with the controller, the timer configured to activate and deactivate the controller at predefined periods of time.

11. An apparatus for cleaning a pet's paw, the apparatus comprising:
a) a cylindrical container for holding water and/or cleaning fluid, the cylindrical container having a top opening and one or more water level markings;
b) a cap configured to cover the top opening of the cylindrical container, the cap further including an orifice configured to accept insertion of the pet's paw while preventing water and/or cleaning fluid from exiting the cap;
c) a cylindrical brush element located within the cylindrical container, the brush element including a plurality of bristles that extend towards a central longitudinal axis, wherein an interior volume of the cylindrical brush element is configured to accept the pet's paw;
d) a base for holding the cylindrical container, the base including a handle;
e) an electrical motor configured to interface with the cylindrical brush element so as to spin the cylindrical brush element to clean the pet's paw, the electrical motor further configured to spin the cylindrical brush element in a clockwise and counterclockwise direction, to move the cylindrical brush element upwards and downwards along the central longitudinal axis, and to move the cylindrical brush element radially away from and towards the central longitudinal axis;
f) a controller configured to control the electrical motor, the controller configured to activate the electrical motor to spin in the clockwise and counterclockwise directions, to move upwards and downwards along the central longitudinal axis, and to move radially away from and towards the central longitudinal axis; and
g) one or more ultraviolet (UV) lights located within the cylindrical container, the one or more UV lights configured to sanitize by emitting UV light.

12. The apparatus of claim 11, wherein the cylindrical container is a clear container.

13. The apparatus of claim 12, wherein the cap comprises a plurality of flaps that extend towards a central point of the cap.

14. The apparatus of claim 13, wherein the plurality of bristles are composed of a foam material.

15. The apparatus of claim 14, wherein the base comprises a bottom side that is wider than a top side.

16. The apparatus of claim 15, wherein the electrical motor is conductively coupled to a rechargeable battery.

17. The apparatus of claim 16, wherein the controller is configured to switch between activating the electrical motor to spin in the clockwise and counterclockwise directions, activating the electrical motor to move upwards and downwards along the central longitudinal axis, and activating the electrical motor to move radially away from and towards the central longitudinal axis.

18. The apparatus of claim 17, wherein the handle comprises a shaft that extends upwards parallel to the central longitudinal axis.

19. The apparatus of claim 18, further comprising an external button on the base, the button configured to activate and deactivate the controller.

20. The apparatus of claim 19, further comprising a timer communicatively coupled with the controller, the timer configured to activate and deactivate the controller at predefined periods of time.
